(19) United States
(12) Patent Application Publication
(10) Pub. No.: US 2008/0206851 A1
(43) Pub. Date: Aug. 28, 2008

(54) Methods and compositions for RNA synthesis

(76) Inventors: Douglas J. Dellinger, Boulder, CO (US); Geraldine F. Dellinger, Boulder, CO (US); Marvin H. Caruthers, Boulder, CO (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/713,424

(22) Filed: Feb. 28, 2007

(51) Int. Cl. C07H 21/02; C12M 1/34

(52) U.S. Cl. 435/287.2; 536/22.1; 536/25.31

(57) ABSTRACT

In some embodiments, the present disclosure relates to new phosphoramidite compositions, that have Silyl-containing carbonate or thiocarbonate or ether as 5′-hydroxyl protecting groups useful fo the syntheis of RNA, and in particular for the synthesis of long sequences of RNA (e.g., >50 mer). In some embodiments, there are provided methods for simultaneous oxidation of the internucleoside phosphate triester linkages and removal of the 5′-hydroxyl-protecting group, making this process a new 2-step RNA synthesis, that involves the use of peroxyanions in combination with fluoride anions.

### BACKGROUND

Solid phase chemical synthesis of oligonucleotides is routinely performed using protected nucleoside phosphoramidites. S. L. Beaucage et al. (1981) Tetrahedron Lett. 22:1859. In this approach, the 3′-hydroxyl group of an initial 5′-protected nucleoside is first covalently attached to the polymer support. R. C. Pless et al. (1975) Nucleic Acids Res. 2:773 (1975). Synthesis of the oligonucleotide then proceeds by deprotection of the 5′-hydroxyl group of the attached nucleoside, followed by coupling of an incoming nucleoside-3′-phosphoramidite to the deprotected hydroxyl group. M. D. Matteucci et a. (1981) J. Am. Chem. Soc. 103:3185. The resulting phosphite triester is finally oxidized to a phosphorotriester to complete the internucleotide bond. R. L. Letsinger et al. (1976) J. Am. Chem. Soc. 9:3655. The steps of deprotection, coupling and oxidation are repeated until an oligonucleotide of the desired length and sequence is obtained. This process is illustrated schematically in FIG. 1 (wherein “B” represents a purine or pyrimidine base, “DMT” represents dimethoxytrityl and “iPR” represents isopropyl).

The chemical group conventionally used for the protection of nucleoside 5′-hydroxyls is dimethoxytrityl (“DMT”), which is removable with acid. H. G. Khorana (1968) Pure Appl. Chem. 17:349; M. Smith et al. (1962) J. Am. Chem. Soc. 84:430. This acid-labile protecting group provides a number of advantages for working with both nucleosides and oligonucleotides. For example, the DMT group can be introduced onto a nucleoside regioselectively and in high yield. E. I. Brown et al. (1979) Methods in Enzymol. 6:109. Also, the lipophilicity of the DMT group greatly increases the solubility of nucleosides in organic solvents, and the carbocation resulting from acidic deprotection gives a strong chromophore, which can be used to indirectly monitor coupling efficiency. M. D. Matteucci et al. (1980) Tetrahedron Lett. 21:719. In addition, the hydrophobicity of the group can be used to aid separation on reverse-phase HPLC. C. Becker et al. (1985) J. Chromatogr. 326:219.

However, use of DMT as a hydroxyl-protecting group in oligonucleotide synthesis is also problematic. The N-glycosidic linkages of oligodeoxyribonucleotides are susceptible to acid catalyzed cleavage (N. K. Kochetkov et al., Organic Chemistry of Nucleic Acids (New York: Plenum Press, 1972)), and even when the protocol is optimized, recurrent removal of the DMT group with acid during oligonucleotide synthesis results in depurination. H. Shaller et al. (1963) J. Am. Chem. Soc. 8:3821. The N-6-benzoyl-protected deoxyadenosine nucleotide is especially susceptible to glycosidic cleavage, resulting in a substantially reduced yield of the final oligonucleotide. J. W. Efcavitch et al. (1985) Nucleosides & Nucleotides 4:267. Attempts have been made to address the problem of acid-catalyzed depurination utilizing alternative mixtures of acids and various solvents; see, for example, E. Sonveaux (1986) Bioorganic Chem. 4:274. However, this approach has met with limited success. L. J. McBride et al. (1986) J. Am. Chem. Soc. 108:2040.

Conventional synthesis of oligonucleotides using DMT as a protecting group is problematic in other ways as well. For example, cleavage of the DMT group under acidic conditions gives rise to the resonance-stabilized and long-lived bis(p-anisyl)phenylmethyl carbocation. P. T. Gilham et al. (1959) J. Am. Chem. Soc. 81:4647. Protection and deprotection of hydroxyl groups with DMT are thus readily reversible reactions, resulting in side reactions during oligonucleotide synthesis and a lower yield than might otherwise be obtained. To circumvent such problems, large excesses of acid are used with DMT to achieve quantitative deprotection. As bed volume of the polymer is increased in larger scale synthesis, increasingly greater quantities of acid are required. The acid-catalyzed depurination which occurs during the synthesis of oligonucleotides is thus increased by the scale of synthesis. M. H. Caruthers et al., in Genetic Engineering: Principles and Methods, J. K. Setlow et al., Eds. (New York: Plenum Press, 1982).

Considerable effort has been directed to developing 5′-O-protecting groups which can be removed under non-acidic conditions. For example, R. L. Letsinger et al. (1967) J. Am. Chem. Soc. 89:7147, describe use of a hydrazine-labile benzoyl-propionyl group, and J. F. M. deRooij et al. (1979) Real. Track. Chain. Pays-Bas. 9:537, describe using the hydrazine-labile levulinyl ester for 5′-OH protection (see also S. Iwai et al. (1988) Tetrahedron Lett. 22:5383; and S. Iwai et al. (1988) Nucleic Acids Res. 16:9443). However, the cross-reactivity of hydrazine with pyrimidine nucleotides (as described in F. Baron et al. (1955) J. Chem. Soc. 2855 and in V. Habermann (1962) Biochem. Biophys. Acta 55:999), the poor selectivity of levulinic anhydride and hydrazine cleavage of N-acyl protecting groups (R. L. Letsinger et al. (1968), Tetrahedron Lett. 22:2621) have made these approaches impractical. H. Seliger et al. (1985), Nucleosides & Nucleotides 4:153, describes the 5′-O-phenyl-azophenyl carbonyl (“PAPco”) group, which is removed by a two-step procedure involving transesterification followed by β-elimination; however, unexpectedly low and non-reproducible yields resulted. Fukuda et al. (1988) Nucleic Acids Res. Symposium Ser.19, 13, and C. Lehmann et al. (1989) Nucleic Acids Res. 17:2389, describe application of the 9-fluorenylmethylcarbonate (“Fmoc”) group for 5′-protection. C. Lehmann et al. (1989) report reasonable yields for the synthesis of oligonucleotides up to 20 nucleotides in length. The basic conditions required for complete deprotection of the Fmoc group, however, lead to problems with protecting group compatibility. Similarly, R. L. Letsinger et al. (1967), J. Am. Chem. Soc. 32:296, describe using the p-nitrophenyloxycarbonyl group for 5′-hydroxyl protection. In all of the procedures described above utilizing base-labile 5′-O-protecting groups, the requirements of high basicity and long deprotection times have severely limited their application for routine synthesis of oligonucleotides.

Scaringe et. al. developed a set of 5′- and 2′-protecting groups that overcome the problems associated with use of 5′-DMT. This method uses a 5′-silyloxy protecting group (U.S. Pat. Nos. 5,889,136; 6,111,086; 6,008,400; and 6,590,093), which require silicon-specific fluoride ion nucleophiles to be removed, in conjugation with the use of optimized 2′-orthoester protecting groups, such as, for example, O-bis(2-acetyl-ethoxy)methyl (ACE) orthoester protecting groups, and 2′-bis(2hydroxyethyl)methyl orthoester protecting groups (2′-EG). This chemistry requires atypical nucleoside protecting groups and custom synthesized monomers, so it cannot utilize many commercially available standard monomers. It is noteworthy to recognize that while the ACE RNA chemistry uses a 5′-silyl containing protecting group, this group is linked via a silylether linkage meaning that the 5′oxygen is directly linked to the silicon atom, making the synthesis of the monomer more difficult because of its non-regiospecific introduction into the nucleoside. It has also been observed that this kind of silylether protecting group requires the presence of bulky aryl substituents on the silicon to render the removal of the silyl group more efficient and more complete.

The problems associated with the use of DMT are exacerbated in solid phase oligonucleotide synthesis where “microscale” parallel reactions are taking place on a very dense, packed surface. Applications in the field of genomics and high throughput screening have fueled the demand for precise chemistry in such a context. Thus, increasingly stringent demands are placed on the chemical synthesis cycle as it was originally conceived, and the problems associated with conventional methods for synthesizing oligonucleotides are rising to unacceptable levels in these expanded applications.

The foregoing methods of preparing polynucleotides are well known and described in detail, for example, in Caruthers (1985) Science 230: 281-285; Itakura et al., Arm. Rev. Biochem. 53: 323-356; Hunkapillar et al. (1984) Nature 310: 105-110; and in “Synthesis of Oligonucleotide Derivatives in Design and Targeted Reaction of Oligonucleotide Derivatives”, CRC Press, Boca Raton, Fla., pages 100 et seq.; U.S. Pat. No. 4,458,066; U.S. Pat. No. 4,500,707; U.S. Pat. No. 5,153,319; U.S. Pat. No. 5,869,643; EP 0294196, and elsewhere. The phosphoramidite and phosphite triester approaches are most broadly used, but other approaches include the phosphodiester approach, the phosphotriester approach and the H-phosphonate approach.

Oligonucleotides may be useful as diagnostic or screening tools, for example, on polynucleotide arrays. Such arrays include regions of usually different sequence polynucleotides arranged in a predetermined configuration on a substrate. These regions (sometimes referenced as “features”) are positioned at respective locations (“addresses”) on the substrate. The arrays, when exposed to a sample, will exhibit an observed binding pattern. This binding pattern can be detected upon interrogating the array. For example all polynucleotide targets (for example, DNA and RNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Polynucleotide arrays can be fabricated by depositing previously obtained polynucleotides onto a substrate, or by in situ synthesis methods. The in situ fabrication methods include those described in WO 98/41531 and the references cited therein. The in situ method for fabricating a polynucleotide array typically follows, at each of the multiple different addresses at which features are to be formed, the same conventional iterative sequence used in forming polynucleotides on a support by means of known chemistry.

In the case of array fabrication, different monomers may be deposited at different addresses on the substrate during any one iteration so that the different features of the completed array will have different desired polynucleotide sequences. One or more intermediate further steps may be required in each iteration, such as the conventional oxidation and washing steps.

Each iteration of the foregoing conventional sequence can have a very high yield (over 90%), with each step being relatively rapid (requiring less than a minute). Thus, the foregoing conventional sequence is ideal for preparing a particular polyribonucleotide on a packed column. Whether the preparation requires four or five minutes is usually not great concern. However, when it is desired to mass produce a polyribonucleotide array with hundreds or more typically, thousands, of features each carrying different polyribonucleotides requiring ten, twenty or more cycles, the time taken for each step in each cycle at each feature becomes much more important. Furthermore, each step in the cycle requires its own solutions and appropriate system of delivery to the substrate during in situ array fabrication, which complicates an in situ array fabrication apparatus and can lead to more waste. It would be desirable then, to provide a means of fabricating an array by the in situ process with a simplified synthesis cycle requiring requiring fewer steps and/or less time to complete each cycle. It would further be desirable if the number of solutions required for each cycle could be reduced.

### SUMMARY

Applicants have found new monomer compositions, in particular new 5′-hydroxyl protecting groups and new 3′-hydroxyl protecting groups. For example, in some embodiments, such hydroxyl-protecting groups comprise a silyl group and an elimination group (ELgp). In some embodiments, such hydroxyl-protecting groups also comprise a carbonate (or thiocarbonate) linking group (Fgp). It has been found that these protecting groups allow a very efficient coupling reaction when synthesizing RNA. Corresponding compositions, kits and methods are provided.

In some aspects, the present disclosure provides novel methods for synthesizing oligoribonucleotides, wherein the method has numerous advantages relative to prior methods such as those discussed above. In some embodiments, the novel methods involve the use of neutral or mildly basic conditions to remove hydroxyl-protecting groups, such that acid-induced depurination is avoided. In addition, the reagents used provide for irreversible deprotection, significantly reducing the likelihood of unwanted side reactions and increasing the overall yield of the desired product. In some embodiments, the methods provide for simultaneous oxidation of the internucleoside phosphite triester linkage and removal of the hydroxyl-protecting group, eliminating the extra step present in conventional processes for synthesizing oligoribonucleotides. The methods are useful in carrying out either 3′-to-5′ synthesis or 5′-to-3′ synthesis. Because of the far more precise chemistry, the methods readily lend themselves to the highly parallel, microscale synthesis of oligoribonucleotides.

In some embodiments, the use of carbonate or thiocarbonates or ether functionalities attached to an “Eliminating Group” (ELgp) linked to a silyl moiety as new protecting groups for the 5′-hydroxyl (or 3′-hydroxyl), that are very efficiently removed by fluoride anions is disclosed herein as new compositions and methods for RNA synthesis, particularly as a new 2-step chemistry that allows for long RNA synthesis.

In some embodiments, there are provided herein methods of synthesizing a sequence of RNA, the methods comprising the steps of: (a) condensing a 3′-OH or a 5′-OH group of a support bound ribonucleoside or oligoribonucleotide with a monomeric ribonucleoside phosphoramidite having a Silyl-ELgp-protected hydroxyl group, to provide an intermediate in which the support-bound ribonucleoside or oligoribonucleotide is bound to the monomeric oligoribonucleoside through a phosphate triester linkage; (b) treating the intermediate provided in step (a) with a deprotecting reagent effective to convert the Silyl-ELgp-protected hydroxyl group to a free hydroxyl moiety; and, c) repeating steps (a)-(b) until the desired sequence of RNA is obtained. Non-limiting examples of the deprotecting reagent include: HF/pyridine; HF/TEA; HF/TEMED; and TBAF.

In some embodiments, there are provided herein methods of synthesizing a sequence of RNA, the method comprising the steps of: (a) condensing a 3′-OH or a 5′-OH group of a support bound ribonucleoside or oligoribonucleotide with a monomeric ribonucleoside phosphoramidite having a Silyl-ELgp-protected hydroxyl group, to provide an intermediate in which the support-bound ribonucleoside or oligoribonucleotide is bound to the monomeric ribonucleoside through a phosphate triester linkage, and (b) treating the intermediate provided in step (a) with a deprotecting reagent effective to convert the Silyl-ELgp-protected hydroxyl group to a free hydroxyl moiety and simultaneously oxidize the phosphate triester linkage to give a phosphotriester linkage. Non-limiting examples of the deprotecting reagent comprise: HF/TEA/ROOH; HF/TEMED/ROOH; and TBAF/ROOH.

Additional advantages and novel features of the invention will be set forth in part in the description which follows, and in part will become apparent to those skilled in the art upon examination of the following, or may be learned by practice of the methods.

### BRIEF DESCRIPTION OF THE DRAWINGS

FIG. 1 schematically illustrates conventional 3′-to-5′ oligonucleotide synthesis using DMT as a 5′-OH protecting group, and separate deprotection and oxidation steps.

FIG. 2 schematically illustrates 3′-to-5′ oligoribonucleotide synthesis using methods of the present disclosure.

FIGS. 3A and 3B compare the conventional deprotection reaction in which DMT is used as a hydroxyl-protecting group (FIG. 3A) and the deprotection reaction (Scheme I) in which reagents of the present disclosure are employed (FIG. 3B).

FIG. 4 is a perspective view of a substrate bearing multiple arrays, as may be produced by methods and apparatuses of the present disclosure.

FIG. 5 is an enlarged view of a portion of FIG. 4 showing some of the identifiable individual regions (or “features”) of a single array of FIG. 5.

FIG. 6 is an enlarged cross-section of a portion of FIG. 5.

FIG. 7 is a schematic view of some embodiments of apparatuses of the present disclosure.

### DESCRIPTION

In general aspects, it is disclosed herein that rapid and selective removal of suitable 5′-OH (or 3′-OH) protecting groups following phosphoramidite condensation can be achieved by employing nucleophiles, and particularly peroxy anions, that exhibit an “alpha effect” under neutral or mildly basic conditions. Rapid and selective deprotection can be achieved under such conditions by employing silyl-ELgp containing protecing groups, as described herein, for 5′-OH (or 3′-OH) protection. Deprotection of ribonucleosides having a silyl-ELgp protecting group using peroxy anions can be conducted in aqueous solution, at neutral or mild pH, resulting in quantitative removal of the silyl-ELgp protecing group and concomitant and quantitative oxidation of the internucleotide phosphite triester bond. Oligoribonucleotides synthesized using the novel methodology can be isolated in high yield.

The term “alpha effect,” as in an “alpha effect” nucleophilic deprotection reagent, is used to refer to an enhancement of nucleophilicity that is found when the atom adjacent a nucleophilic site bears a lone pair of electrons. As the term is used herein, a nucleophile is said to exhibit an “alpha effect” if it displays a positive deviation from a BrØnsted-type nucleophilicity plot. S. Hoz et al. (1985) Israel J. Chem. 26:313. See also, J. D. Aubort et al. (1970) Chem. Comm. 1378; J. M. Brown et al. (1979) J. Chem. Soc. Chem. Comm. 171; E. Buncel et al.(1982) J. Am. Chem. Soc. 104:4896; J. O. Edwards et al. (1962) J. Amer. Chem. Soc. 84:16; J. D. Evanseck et al. (1987) J. Am. Chem Soc. 109:2349. The magnitude of the alpha effect is dependent upon the electrophile which is paired with the specific nucleophile. J. E. Mclsaac, Jr. et al. (1972), J. Org. Chem. 31:1037. Peroxy anions are examples of nucleophiles which exhibit strong alpha effects.

In some aspects, the present disclosure provides for efficient solid-phase synthesis of oligoribonucleotides of lengths 25 nucleotides or more. Treatment using an alpha effect nucleophile as presently described for removal of silyl-ELgp-protecting groups is irreversible, resulting in fragmentation of the protecting group. Moreover, because such treatment results in concomitant oxidation of the internucleotide bond and substantial removal of exocyclic amine protecting groups, the disclosed methods can obviate the need for a separate oxidation step and a postsynthesis deprotection step to remove any exocyclic amine protecting groups that may be used.

In some aspects, there are provided methods for making an oligoribonucleotide array made up of array features each presenting a specified oligoribonucleotide sequence at an address on an array substrate, by first treating the array substrate to protect the hydroxyl moieties on the derivatized surface from reaction with phosphoramidites, then carrying out the steps of (a) applying droplets of an alpha effect nucleophile to effect deprotection of hydroxyl moieties at selected addresses, and (b) flooding the array substrate with a medium containing a selected silyl-ELgp protected phosphoramidite to permit coupling of the selected phosphoramidite onto the deprotected hydroxyl moieties at the selected addresses, and repeating the steps (a) and (b) to initiate and to sequentially build up oligoribonucleotides having the desired sequences at the selected addresses to complete the array features. In a variation on the aforementioned method, the droplets applied may comprise the protected phosphoramidite, and the alpha effect nucleophile may be used to flood the array substrate.

In some embodiments of array construction methods according to the disclosure, the deprotection reagents are aqueous, allowing for good droplet formation on a wide variety of array substrate surfaces. Moreover, because the selection of features can employ aqueous media, small-scale discrete droplet application onto specified array addresses can be carried out by adaptation of techniques for reproducible fine droplet deposition from printing technologies. Further, as noted above, the synthesis reaction provides irreversible deprotection, and thus quantitative removal of protecting groups within each droplet may be achieved. The phosphoramidite reactions can be carried out in bulk, employing an excess of the phosphoramidite during the coupling step (b), allowing for exclusion of water by action of the excess phosphoramidite as a desiccant.

It is to be understood that unless otherwise indicated, this disclosure is not limited to specific reagents, reaction conditions, synthetic steps, or the like, as such may vary. It is also to be understood that the terminology used herein is for the purpose of describing particular embodiments only and is not intended to be limiting.

### Definitions and Nomenclature

It is to be understood that unless otherwise indicated, this disclosure is not limited to specific reagents, reaction conditions, synthetic steps, or the like, as such may vary. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise. Thus, for example, reference to “a protecting group” includes combinations of protecting groups, reference to “a nucleoside” includes combinations of nucleosides, and the like. Similarly, reference to “a substituent” as in a compound substituted with “a substituent” includes the possibility of substitution with more than one substituent, wherein the substituents may be the same or different.

In this specification and in the claims which follow, reference will be made to a number of terms which shall be defined to have the following meanings:

It will be appreciated that, as used herein, the terms “nucleoside” and “nucleotide” will include those moieties which contain not only the known purine and pyrimidine bases, but also modified purine and pyrimidine bases and other heterocyclic bases which have been modified (these moieties are sometimes referred to herein, collectively, as “purine and pyrimidine bases and analogs thereof”). Such modifications include methylated purines or pyrimidines, acylated purines or pyrimidines, and the like.

A “nucleotide” or a “nucleotide moiety” refers to a subunit of a nucleic acid (whether DNA or RNA) which may include, but is not limited to, a phosphate group, a sugar group and a nitrogen containing base. Other groups (e.g., protecting groups) can be attached to any component(s) of a nucleotide or nucleotide moiety.

A “nucleoside” or a “nucleoside moiety” refers to a nucleic acid subunit including a sugar group and a nitrogen containing base. Other groups (e.g., protecting groups) can be attached to either or both components of a nucleoside or nucleoside moiety. It will be appreciated that, as used herein, the terms “nucleoside” and “nucleotide” will include those moieties which contain not only the known purine and pyrimidine bases, but also modified purine and pyrimidine bases and other heterocyclic bases which have been modified (these moieties are sometimes referred to herein, collectively, as “purine and pyrimidine bases and analogs thereof”). Such modifications include methylated purines or pyrimidines, acylated purines or pyrimidines, and the like.

As used herein, the term “oligonucleotide” shall be generic to polydeoxynucleotides (containing 2-deoxy-D-ribose), to polyribonucleotides (containing D-ribose), to any other type of polynucleotide which is an N-glycoside of a purine or pyrimidine base, and to other polymers containing nonnucleotidic backbones, providing that the polymers contain nucleobases in a configuration which allows for base pairing and base stacking, such as is found in DNA and RNA. The terms “oligonucleotide” and “polynucleotide” are often used interchangeably, consistent with the context of the sentence and paragraph in which they are used in.

The term “nitrogen-containing base” includes not only the naturally occurring purine and pyrimidine bases, e.g., adenine (A), thymine (T), cytosine (C), guanine (G), or uracil (U), but also modified purine and pyrimidine bases and other heterocyclic bases which have been modified. Such bases include, e.g., diaminopurine, inosine, 3-nitropyrrole, 5-nitroindole, alkylated purines or pyrimidines, acylated purines or pyrimidines, thiolated purines or pyrimidines, and the like, or bases with the addition of a protecting group such as acetyl, difluoroacetyl, trifluoroacetyl, isobutyryl, benzoyl, 9-fluorenylmethoxycarbonyl, phenoxyacetyl, dimethylformamidine, N,N-diphenyl carbamate, or the like. The purine or pyrimidine base may also be an analog of the foregoing; suitable analogs will be known to those skilled in the art. Common analogs include, but are not limited to, 1-methyladenine, 2-methyladenine, N⁶-methyladenine, N⁶-isopentyladenine, 2-methylthio-N⁶-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanilie, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, 2-deoxyinosine, 1-methylinosine, hypoxanthine, xanthine, 2-aminopurine, 6-hydroxyaminopurine, 6-thiopurine, and 2,6-diaminopurine.

An “internucleotide bond” or “nucleotide bond” refers to a chemical linkage between two nucleoside moieties, such as the phosphodiester linkage in nucleic acids found in nature, or linkages well known from the art of synthesis of nucleic acids and nucleic acid analogues. An internucleotide bond may include a phospho or phosphite group, and may include linkages where one or more oxygen atoms of the phospho or phosphite group are either modified with a substituent or replaced with another atom, e.g., a sulfur atom, or the nitrogen atom of a mono- or di-alkyl amino group.

A “group” includes both substituted and unsubstituted forms. Typical substituents include one or more lower alkyl, amino, imino, amido, alkylamino, arylamino, alkoxy, aryloxy, thio, alkylthio, arylthio,or aryl, or alkyl; aryl, alkoxy, thioalkyl, hydroxyl, amino, amido, sulfonyl, thio, mercapto, imino, halo, cyano, nitro, nitroso, azido, carboxy, sulfide, sulfone, sulfoxy, phosphoryl, silyl, silyloxy, and boronyl, or optionally substituted on one or more available carbon atoms with a nonhydrocarbyl substituent such as cyano, nitro, halogen, hydroxyl, sulfonic acid, sulfate, phosphonic acid, phosphate, phosphonate, or the like. Substituents can be chosen so as not to substantially adversely affect reaction yield (for example, not lower it by more than 20% (or 10%, or 5%, or 1%) of the yield otherwise obtained without a particular substituent or substituent combination). In some embodiments, for any group in this disclosure, each substituent contains up to 40, 35, 30, 25, 20, 18, 16, 14, 12, 11, 10, 9, 8, 7, 6, 5, 4 or 3 carbon atoms. Overall, in some embodiments, the total number of carbon atoms in all the substituents for any group is no more than 80, 75, 70, 65, 60, 55, 50, 45, 40, 35, 30, 25, 20, 18, 16, 14, 12, 11, 10, 9, 8, 7, 6, 5, 4 or 3.

A “phospho” group includes a phosphodiester, phosphotriester, and H-phosphonate groups. In the case of either a phospho or phosphite group, a chemical moiety other than a substituted 5-membered furyl ring may be attached to O of the phospho or phosphite group which links between the furyl ring and the P atom.

A “protecting group” is used in the conventional chemical sense as a group, which reversibly renders unreactive a functional group under certain conditions of a desired reaction, as taught, for example, in Greene, et al., “Protective Groups in Organic Synthesis,” John Wiley and Sons, Second Edition, 1991. After the desired reaction, protecting groups may be removed to deprotect the protected functional group. All protecting groups should be removable (and hence, labile) under conditions which do not degrade a substantial proportion of the molecules being synthesized. In contrast to a protecting group, a “capping group” permanently binds to a segment of a molecule to prevent any further chemical transformation of that segment. It should be noted that the functionality protected by the protecting group may or may not be a part of what is referred to as the protecting group.

A “hydroxyl protecting group” or “O-protecting group” refers to a protecting group where the protected group is a hydroxyl. A “reactive-site hydroxyl” is the terminal 5′-hydroxyl during 3′-5′ polynucleotide synthesis, or the 3′-hydroxyl during 5′-3′ polynucleotide synthesis. An “acid-labile protected hydroxyl” is a hydroxyl group protected by a protecting group that can be removed by acidic conditions. Similarly, an “alkaline-labile protecting group” is a protecting group that can be removed by alkaline conditions.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

The term “hydrocarbyl” refers to alkyl, alkenyl or alkynyl.

The term “alkyl” refers to a saturated straight chain, branched, or cyclic hydrocarbon group of 1 to 30 carbon atoms. An alkyl typically contains 1-24, 1-20, 1-16, 1-12, 1-10, 1-8, 1-6 or 1-4 carbon atoms. A “lower alkyl” is an alkyl with 1 to 6 carbon atoms. Exemplary alkyls include methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. Lower alkyls include, for example, methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The term “cycloalkyl” refers to cyclic alkyl groups such as cyclopropyl, cyclobutyl, cyclopentyl, cyclohexyl, cycloheptyl and cyclooctyl. Cycloalkyls typically have from about 3 to about 10 carbon atoms in their ring structure, and alternatively about 5, 6 or 7 carbons in the ring structure.

The term “alkenyl” refers to a branched, unbranched, or cyclic hydrocarbon group of 2 to 30 carbon atoms containing at least one double bond, such as ethenyl, vinyl, allyl, octenyl, decenyl, and the like. An alkenyl typically contain 2-24, 2-20, 2-16, 2-12, 2-10, 2-8, 2-6 or 2-4 carbon atoms. The term “lower alkenyl” refers to an alkenyl group of two to six carbon atoms, and specifically includes vinyl and allyl by way of example. The term “cycloalkenyl” refers to cyclic alkenyl groups.

The term “alkynyl” refers to a branched or unbranched hydrocarbon group of 2 to 30 carbon atoms containing at least one triple bond, such as acetylenyl, ethynyl, n-propynyl, isopropynyl, n-butynyl, isobutynyl, t-butynyl, octynyl, decynyl and the like. An alkynyl typically contain 2-24, 2-20, 2-16, 2-12, 2-10, 2-8, 2-6 or 2-4 carbon atoms. The term “lower alkynyl” refers to an alkynyl group of two to six carbon atoms, and includes, for example, acetylenyl and propynyl. The term “cycloalkynyl” refers to cyclic alkynyl groups.

The term “substituted hydrocarbyl” refers to hydrocarbyl moieties having substituents replacing a hydrogen on one or more carbons of the hydrocarbon backbone. Such substituents may include, for example, a hydroxyl, a halogen, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclic, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CN, and the like. Cycloalkyls may be further substituted with alkyls, alkenyls, alkoxys, alkylthios, aminoalkyls, carbonyl-substituted alkyls, —CN, and the like.

The term “alkoxy” means an alkyl group linked to oxygen: R—O—, wherein R represents the alkyl group. An example is the methoxy group CH₃O—.

The term “aryl” refers to 5-, 6-, and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The term “aryl” also includes polycyclic ring systems having two or more cyclic rings in which two or more carbons are common to two adjoining rings (the rings are “fused rings”) wherein at least one of the rings is aromatic (e.g., the other cyclic rings may be cycloalkyls, cycloalkenyls, cycloalkynyls, aryls, and/or heterocycles). A “lower aryl” contains up to 18 carbons, more preferably up to 14, 12, 10, 8 or 6 carbons.

The aromatic rings may be substituted at one or more ring positions with such substituents as described above for substituted hydrocarbyls, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclic, aromatic or heteroaromatic moieties, —CF₃, —CN, or the like.

The terms “halogen” and “halo” refer to fluorine, chlorine, bromine, or iodine.

The term “heterocycle”, “heterocyclic”, “heterocyclic group” or “heterocyclo” refers to fully saturated or partially or completely unsaturated cyclic groups having at least one heteroatom in at least one carbon atom-containing ring, including aromatic (“heteroaryl”) or nonaromatic (for example, 3 to 13 member monocyclic, 7 to 17 member bicyclic, or 10 to 20 member tricyclic ring systems). Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system. The rings of multi-ring heterocycles may be fused, bridged and/or joined through one or more spiro unions. Nitrogen-containing bases are examples of heterocycles. Other examples include piperidinyl, morpholinyl and pyrrolidinyl.

The terms “substituted heterocycle”, “substituted heterocyclic”, “substituted heterocyclic group” and “substituted heterocyclo” refer to heterocycle, heterocyclic, and heterocyclo groups substituted with one or more groups preferably selected from alkyl, substituted alkyl, alkenyl, oxo, aryl, substituted aryl, heterocyclo, substituted heterocyclo, carbocyclo (optionally substituted), halo, hydroxy, alkoxy (optionally substituted), aryloxy (optionally substituted), alkanoyl (optionally substituted), aroyl (optionally substituted), alkylester (optionally substituted), arylester (optionally substituted), cyano, nitro, amido, amino, substituted amino, lactam, urea, urethane, sulfonyl, and the like, where optionally one or more pair of substituents together with the atoms to which they are bonded form a 3 to 7 member ring.

When used herein, the terms “hemiacetal”, “thiohemiacetal”, “acetal”, and “thioacetal”, are recognized in the art, and refer to a chemical moiety in which a single carbon atom is geminally disubstituted with either two oxygen atoms or a combination of an oxygen atom and a sulfur atom. In addition, when using the terms, it is understood that the carbon atom may actually be geminally disubstituted by two carbon atoms, forming ketal, rather than acetal, compounds.

The term “electron-withdrawing group” is art-recognized, and refers to the tendency of a substituent to attract valence electrons from neighboring atoms (i.e., the substituent is electronegative with respect to neighboring atoms). A quantification of the level of electron-withdrawing capability is given by the Hammett sigma constant. This well known constant is described in many references, for instance, March, Advanced Organic Chemistry 251-59, McGraw Hill Book Company, New York, (1977). Exemplary electron-withdrawing groups include nitro, acyl, formyl, sulfonyl, trifluoromethyl, cyano, chloride, and the like.

The term “electron-donating group” is art-recognized, and refers to the tendency of a substituent to repel valence electrons from neighboring atoms (i.e., the substituent is less electronegative with respect to neighboring atoms). Exemplary electron-donating groups include amino, methoxy, alkyl (including C₁₋₆ alkyl that can have a linear or branched structure), C₄₋₉ cycloalkyl, and the like.

The term “deprotecting simultaneously” refers to a process which aims at removing different protecting groups in the same process and performed substantially concurrently or concurrently. However, as used herein, this term does not imply that the deprotection of the different protecting groups occur at exactly the same time or with the same rate or same kinetics.

An “array” is a collection of separate molecules each arranged in a spatially defined and a physically addressable manner. The number of molecules, or “features,” that can be contained on an array will largely be determined by the surface area of the substrate, the size of a feature and the spacing between features, wherein the array surface may or may not comprise a local background region represented by non-feature area. Generally, arrays can have densities of up to several hundred thousand or more features per cm², and in some embodiments, about 2,500 to about 200,000 features/cm². The features may or may not be covalently bonded to the substrate.

### Oligonucleotide Synthesis Using Silyl-ELgp Protection and Irreversible Nucleophilic Deprotection

A method for making a protecting group more labile to nucleophiles is to incorporate a moiety or moieties that enhances its removal by a fragmentation process that creates thermodynamically stabile fragments, thereby stabilizing the products of the cleavage reaction promoting facile removal of protecting groups under mild pH conditions.

In some aspects, the use of carbonate or thiocarbonates or ether functionalities attached to an “Eliminating Group” (ELgp) linked to a silyl moiety as new protecting groups for the 5′-hydroxyl, that are very efficiently removed by alpha effect nucleophiles, such as, for example, fluoride anions, is disclosed herein as new compositions and methods for RNA synthesis, particularly as a new 2-step chemistry that allows for long RNA synthesis.

In some embodiments, there are proved herein methods for synthesizing an oligoribonucleotide on a solid support, wherein a Silyl-ELgp is used as a hydroxyl-protecting group and an alpha effect nucleophile is used to bring about deprotection. In some embodiments, the novel syntheses are based on a simple, two-step method of (1) coupling a hydroxyl-protected ribonucleoside monomer to a growing oligoribonucleotide chain, and (2) deprotecting the product, under neutral or mildly basic conditions, using an alpha effect nucleophilic reagent that also oxidizes the internucleotide linkage to give a phosphotriester bond. The coupling and deprotection/oxidation steps are repeated as necessary to give an oligoribonucleotide having a desired sequence and length.

In some embodiments of an initial step of the synthesis, then, an unprotected ribonucleoside is covalently attached to a solid support to serve as the starting point for oligoribonucleotide synthesis. The ribonucleoside may be bound to the support through its 3′-hydroxyl group or its 5′-hydroxyl group, but is typically bound through the 3′-hydroxyl group. A second ribonucleoside monomer is then coupled to the free hydroxyl group of the support-bound initial monomer, wherein for 3′-to-5′ oligoribonucleotide synthesis, the second ribonucleoside monomer has a phosphorus derivative such as a phosphoramidite at the 3′ position and a Silyl-ELgp protecting group at the 5′ position, and alternatively, for 5′-to-3′ oligoribonucleotide synthesis, the second ribonucleoside monomer has a phosphorus derivative at the 5′ position and a Silyl-ELgp protecting group at the 3′ position. This coupling reaction gives rise to a newly formed phosphite triester bond between the initial ribonucleoside monomer and the added monomer, with the Silyl-ELgp-protected hydroxyl group intact. In the second step of the synthesis, the Silyl-ELgp group is removed with an alpha effect nucleophile that also serves to oxidize the phosphite triester linkage to the desired phosphotriester.

In some embodiments, for 3′-to-5′ synthesis, support-bound nucleoside monomers are provided having the structure (I)

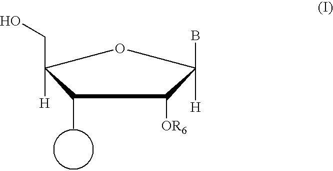

wherein: O represents the solid support or a support-bound oligonucleotide chain and B is a purine or pyrimidine base. The purine or pyrimidine base may be conventional, e.g., adenine (A), thymine (T), cytosine (C), guanine (G) or uracil (U), or a protected form thereof, e.g., wherein the base is protected with a protecting group such as acetyl, difluoroacetyl, trifluoroacetyl, isobutyryl, benzoyl, or the like. The purine or pyrimidine base may also be an analog of the foregoing; suitable analogs will be known to those skilled in the art and are described in the pertinent texts and literature. Common analogs include, but are not limited to, 1-methyladenine, 2-methyladenine, N⁶-methyladenine, N⁶-isopentyladenine, 2-methylthio-N⁶-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine.

In some embodiments, protected monomers to be added have the structure of formula (II)

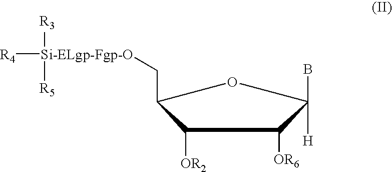

wherein B is a protected or non-protected heterocycle;
each of R₃, R₄, R₅ is independently selected from hydrocarbyls, substituted hydrocarbyls, aryls, and substituted hydrocarbyls;
wherein Fgp is an optional linking group, non-limiting examples of which include oxycarbonyl (O—C(O)) and thiocarbonyl (S—C(O));
wherein ELgp is not oxygen-linked or sulfur-linked to the Si atom;
wherein ELgp is an eliminating group selected from the group consisting of ethylene, substituted ethylene, —(CH₂CH₂O)ₙ—, substituted —(CH₂CH₂O)ₙ—, —(CH₂CH₂O)ₙ—CH₂CH₂—, substituted —(CH₂CH₂O)ₙ—CH₂CH₂— (wherein n is an integer from 1 to 8), and the following functional groups (in the direction from Fgp to Si), and any repeats and combinations of said eliminating groups:

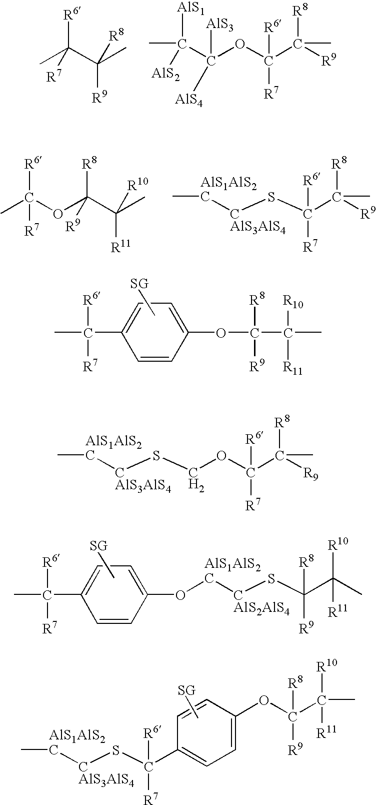

wherein each of R^{6′}, R⁷, R⁸, R⁹, R¹⁰, and R¹¹ is independently selected from the group consisting of H, hydrocarbyls, substituted hydrocarbyls, aryls, and substituted aryls. AIS stands for Allowable Substituents for episulfide formation. The substituents allowable for episulfide formation are known in the art, including, but not limited to, H, halogens, NO₂, CN, lower alkyls, substituted lower alkyls, and the like. SG is one or multiple substituents on the phenyl ring as discussed in the definition of aryls, such as H, halogens, CN, amino, nitro, SO₃, sulfates, nitrates, carbonates, hydrocarbyls, substituted hydrocarbyls, aryls, and substituted aryls, including one or more fused ring. The total number of the repeats and combinations of the above eliminating groups, if any, is preferably 2 to 8, 2 to 6, 2 to 4, or 2;
wherein ELgp is not oxygen-linked or sulfur-linked to the Si atom; and
wherein R₆ is one of the following structures:

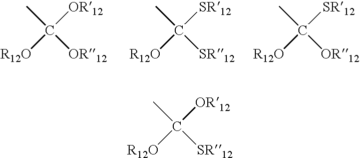

wherein each of R₁₂, R′₁₂, and R″₁₂ are independently selected from lower hydrocarbyls, substituted-lower hydrocarbyls, aryls and substituted hydrocarbyls, wherein R₁₂ is optionally H, and wherein R′₁₂ and R″₁₂ are optionally cyclically connected. In some embodiments, at least one of R₃, R₄, and R₅ is a lower alkyl. In some embodiments, at least one of R₃, R₄, and R₅ is an aryl. In some embodiments, each of R₃, R₄, and R₅ comprises a phenyl group or substituted phenyl group.

Non-limiting examples of Silyl-ELgp-Fgp groups include the following:

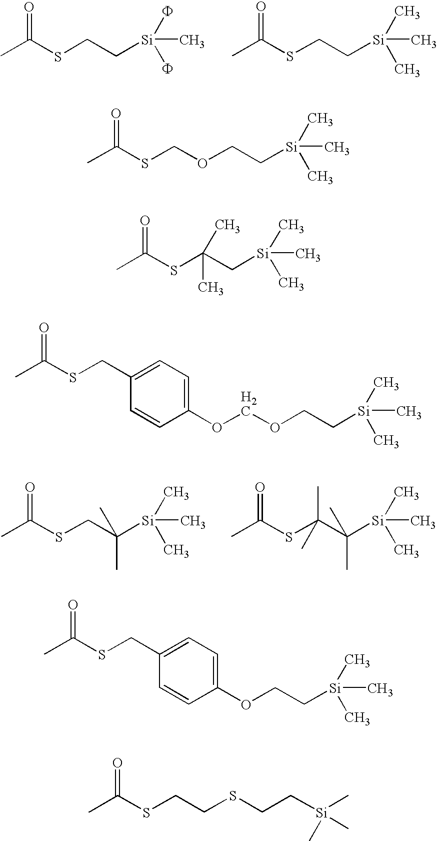

In some embodiments, R₂ is a phosphorus derivative that enables coupling to a free hydroxyl group. In some embodiments, such a phosphorus derivative comprises a phosphoramidite, such that R₂ has the structure (III)

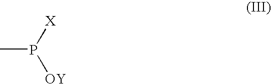

wherein X is —NQ¹Q² in which Q¹ and Q² may be the same or different and are typically selected from the group consisting of alkyl, aryl, aralkyl, alkaryl, cycloalkyl, alkenyl, cycloalkenyl, alkynyl, cycloalkynyl, optionally containing one or more nonhydrocarbyl linkages such as ether linkages, thioether linkages, oxo linkages, amine and imine linkages, and optionally substituted on one or more available carbon atoms with a nonhydrocarbyl substituent such as cyano, nitro, halo, or the like. In some embodiments, each of Y, Q¹ and Q² is independently a hydrocarbyl, substituted hydrocarbyl, heterocycle, substituted heterocycle, aryl or substituted aryl. In some embodiments, Y, Q¹ and Q² are selected from lower alkyls, lower aryls, and substituted lower alkyls and lower aryls (for example, substituted with structures containing up to 18, 16, 14, 12, 11, 10, 9, 8, 7, 6, 5, 4, 3 or 2 carbons). In some embodiments, Q¹ and Q² represent lower alkyl, and can be sterically hindered lower alkyls such as isopropyl, t-butyl, isobutyl, sec-butyl, neopentyl, tert-pentyl, isopentyl, sec-pentyl, and the like. In some embodiments, Q¹ and Q² both represent isopropyl. Q¹ and Q² are optionally cyclically connected. For example, Q¹ and Q² may be linked to form a mono- or polyheterocyclic ring having a total of from 1 to 3, usually 1 to 2 heteroatoms and from 1 to 3 rings. In such a case, Q¹ and Q² together with the nitrogen atom to which they are attached represent, for example, pyrrolidone, morpholino or piperidino. In some embodiments, Q¹ and Q² can have a total of from 2 to 12 carbon atoms. Non-limiting examples of —NQ¹Q² moieties include, but are not limited to, dimethylamine, diethylamine, diisopropylamine, dibutylamine, methylpropylamine, methylhexylamine, methylcyclopropylamine, ethylcyclohexylamine, methylbenzylamine, methylcyclohexylmethylamine, butylcyclohexylamine, morpholine, thiomorpholine, pyrrolidine, piperidine, 2,6-dimethylpiperidine, piperazine, and the like. In some embodiments, moiety “Y” is hydrido or hydrocarbyl, typically alkyl, alkenyl, aryl, aralkyl, or cycloalkyl. In some embodiments, Y represents: lower alkyl; electron-withdrawing β-substituted aliphatic, particularly electron-withdrawing β-substituted ethyl such as β-trihalomethyl ethyl, β-cyanoethyl, β-sulfoethyl, β-nitro-substituted ethyl, and the like; electron-withdrawing substituted phenyl, particularly halo-, sulfo-, cyano- or nitro-substituted phenyl; or electron-withdrawing substituted phenylethyl. In some embodiments, Y represents methyl, β-cyanoethyl, or 4-nitrophenylethyl. In some embodiments, Y is 2-cyanoethyl or methyl, and either or both of Q¹ and Q² is isopropyl.

The coupling reaction can be conducted under standard conditions used for the synthesis of oligonucleotides and conventionally employed with automated oligonucleotide synthesizers. Such methodology will be known to those skilled in the art and is described in the pertinent texts and literature, e.g., in D. M. Matteuci et al. (1980) Tet. Lett. 521:719 and U.S. Pat. No. 4,500,707. The product of the coupling reaction may be represented as structural formula (IV), as follows:

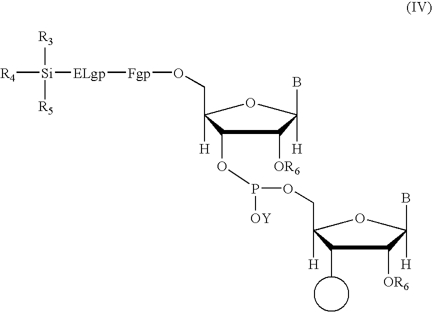

In some embodiments, in a second step of the synthesis, the product (IV) is treated with an “alpha effect” nucleophile in order to remove the Silyl-ELgp protecting group at the 5′ terminus, to generate a 5′-OH. The alpha effect nucleophile also oxidizes the newly formed phosphite triester linkage —O—P(OY)—O— to give the desired phosphotriester linkage

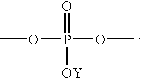

Advantageously, this step can be conducted in an aqueous solution at neutral pH or at a mildly basic pH, depending on the pKa of the nucleophilic deprotection reagent. That is, and as will be explained in further detail below, the pH at which the deprotection reaction is conducted can be above the pKa of the deprotection reagent. Typically, the reaction is conducted at a pH of less than about 10.

In some embodiments, the nucleophilic deprotection reagent that exhibits an alpha effect is a peroxide or a mixture of peroxides, and the pH at which deprotection is conducted is at or above the pKa for formation of the corresponding peroxy anion. The peroxide can be either inorganic or organic. Suitable inorganic peroxides include those of the formula M⁺OOH⁻, where M is any counteranion, including for example H⁺, Li⁺, Na⁺, K⁺, Rb⁺, Cs⁺, or the like. Examples include lithium peroxide or hydrogen peroxide. Examples, of suitable organic peroxides include those of the formula ROOH, where R is selected from the group consisting of alkyl, aryl, substituted alkyl and substituted aryl. In some embodiments, the organic peroxide can have one of the following three general structures (V), (VI) or (VII)

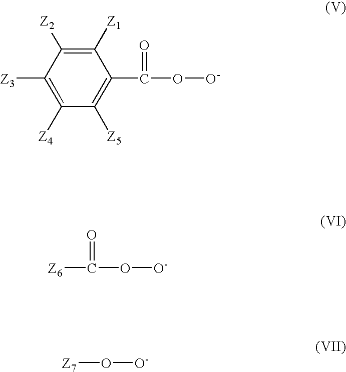

in which Z¹ through Z⁷ are generally hydrocarbyl optionally substituted with one or more nonhydrocarbyl substituents and optionally containing one or more nonhydrocarbyl linkages. Generally, Z¹ through Z⁷ can independently be selected from the group consisting of hydrido, alkyl, aryl, aralkyl, cycloalkyl, cycloalkylalkyl, alkenyl, cycloalkenyl, alkynyl aralkynyl, cycloalkynyl, substituted aralkyl, substituted cycloalkyl, substituted cycloalkylalkyl, substituted alkenyl, substituted cycloalkenyl, substituted alkynyl substituted aralkynyl, substituted cycloalkynyl; t-butyl-hydroperoxide or metachloroperoxybenzoic acid can be particularly suitable. In some embodiments, m-chloroperbenzoic acid (mCPBA) peroxy anion can be used for removal of protecting groups.

The product of this simultaneous deprotection and oxidation step may thus be represented as follows:

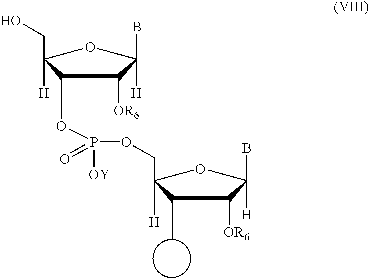

wherein B and Y are as defined earlier herein. In some embodiments, this latter reaction also gives rise to by-products resulting from the elimination of ELgp and Fgp. When present, linking groups such as oxycarbonyl (O—C(O)) or thiocarbonyl (S—C(O)) give rise to CO₂ or C(O)S as by-products, respectively.

The use of a peroxy anion to effect simultaneous removal of the Silyl-ELgp protecting group and oxidation of the internucleotide linkage also removes, to a large extent, exocyclic amine-protecting groups such as acetyl, trifluoroacetyl, difluoroacetyl and trifluoroacetyl moieties. Thus, an added advantage herein is the elimination of a separate post-synthetic reaction step to remove exocyclic amine-protecting groups, as is required with conventional methods of synthesizing oligonucleotides. Elimination of this additional step significantly decreases the time and complexity involved in oligonucleotide synthesis.

An additional advantage of peroxy anions as deprotection reagents herein is that they may be readily activated or inactivated by simply changing pH. That is, the effectiveness of peroxides as nucleophiles is determined by their pKa. In buffered solutions having a pH below the pKa of a particular peroxide, the peroxides are not ionized and thus are non-nucleophilic. To activate a peroxide and render it useful as a deprotection reagent for use herein, the pH is increased above the pKa so that the peroxide is converted to a nucleophilic peroxy anion. Thus, one can carefully control the timing and extent of the deprotection reaction by varying the pH of the peroxide solution used.

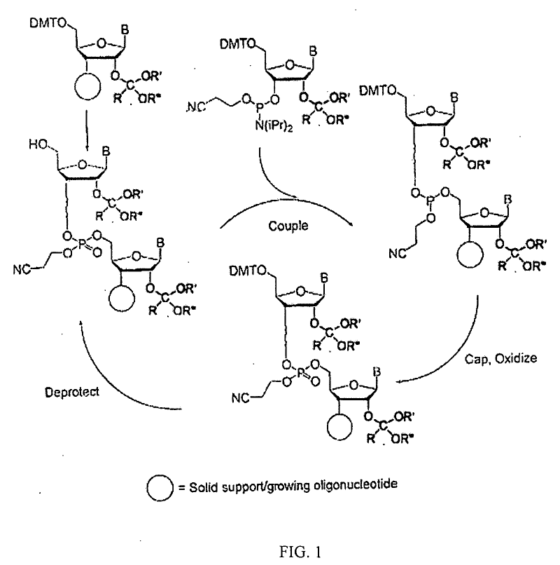

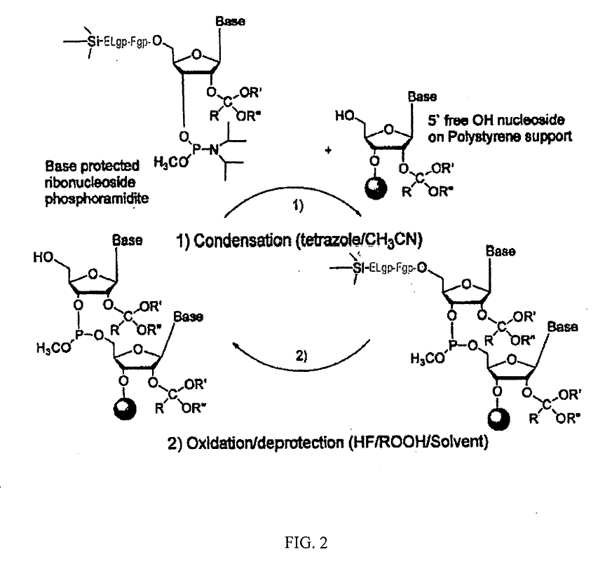

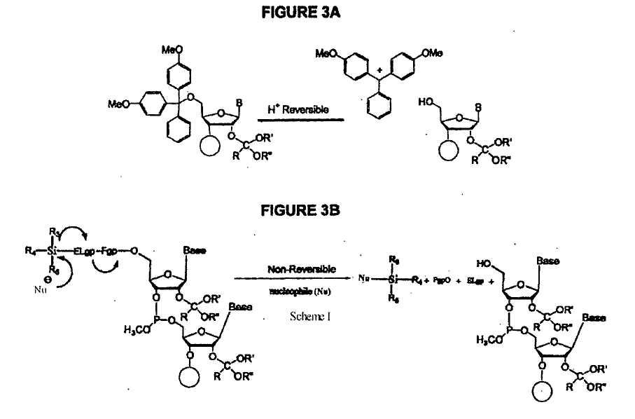

FIG. 2 schematically illustrates 3′-to-5′ synthesis of an oligoribonucleotide using some embodiments of methods disclosed herein. As may be seen, deprotection and oxidation can occur simultaneously. The synthesis may be contrasted with that schematically illustrated in FIG. 1, a conventional method employing DMT protection and separate oxidation and deprotection steps. A further advantage of the present methods is illustrated in FIG. 3. As shown in FIG. 3A, protection and deprotection of hydroxyl groups using DMT is a reversible process, with the DMT cation shown being a relatively stable species. Thus, using DMT as a protecting group can lead to poor yields and unwanted side reactions, insofar as the deprotection reaction is essentially reversible. FIG. 3B illustrates the irreversible deprotection reaction of the present methods, wherein nucleophilic attack of the peroxy anion irreversibly cleaves the Silyl-ELgp moiety. The reaction is not “reversible,” insofar as there is no equilibrium reaction in which a cleaved protecting group could reattach to the hydroxyl moiety, as is the case with removal of DMT.

As mentioned above, the present methods also lend themselves to synthesis in the 5′-to-3′ direction. In such a case, the initial step of the synthetic process involves attachment of a ribonucleoside monomer to a solid support at the 5′ position, leaving the 3′ position available for covalent binding of a subsequent monomer. In this embodiment, i.e., for 5′-to-3′ synthesis, a support-bound ribonucleoside monomer is provided having the structure (IX)

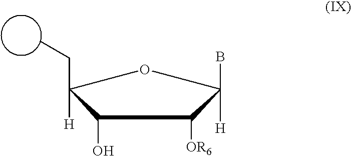

wherein ◯ represents the solid support or a support-bound oligonucleotide-chain and B is a purine or pyrimidine base. The protected monomer to be added has the structure of formula (X)

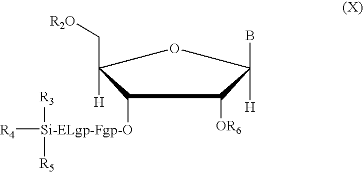

wherein the Silyl-ELgp protecting group is present at the 3′ position and R₂ represents a phosphorus derivative that enables coupling to a free hydroxyl group, and can be a phosphoramidite having the structure (III)

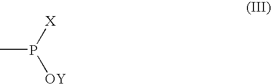

wherein X and Y are as defined earlier. The coupling reaction in which the nucleoside monomer becomes covalently attached to the 3′ hydroxyl moiety of the support bound nucleoside can be conducted under reaction conditions identical to those described for the 3′-to-5′ synthesis. This step of the synthesis gives rise to the intermediate (XI)

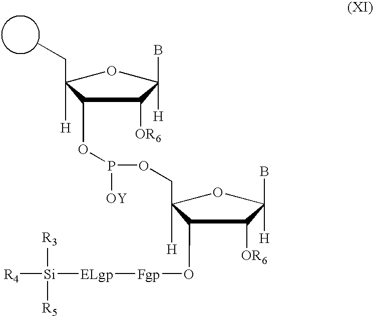

As described with respect to oligonucleotide synthesis in the 3′-to-5′ direction, the coupling reaction is followed by treatment of the product (XI) with an alpha effect nucleophile in order to remove the Silyl-ELgp protecting group at the 3′ terminus, and to oxidize the internucleotide phosphite triester linkage to give the desired phosphotriester linkage.

The two-step process of coupling and deprotection/oxidation is repeated until the oligonucleotide having the desired sequence and length is obtained. Following synthesis, the oligonucleotide may, if desired, be cleaved from the solid support.

The nucleoside monomers as presently described, with the Silyl-ELgp-protecting group, can thus be used to efficiently synthesize oligoribonucleotides. The synthesis can be performed in either direction: from 3′ to 5′ (or from 5′ to 3′) as described above. For example, in the 3′ to 5′ direction, a first ribonucleoside monomer with a 5′-OH and a 3′-protecting group is coupled with a second ribonucleoside monomer having a 3′-phosphoramidite and a 5′-protecting group. The first nucleoside monomer is optionally bound to a solid support. Alternatively, the synthesis can be performed in solution. After the coupling step, in which the 5′-OH and the 3′-phosphoramidite condense to form a phosphite triester linkage and result in a dinucleotide, the dinucleotide is capped/oxidized, and the 5′-protecting group is removed (deprotection). The dinucleotide is then ready for coupling with another nucleoside monomer having a 3′-phosphoramidite and a 5′-protecting group. These steps are repeated until the oligoribonucleotide reaches the desired length and/or sequence, and the 5′-protecting group can be removed as described above.

Thus, in some embodiments, there are provided herein methods of synthesizing a sequence of RNA, the methods comprising the steps of: (a) condensing a 3′-OH or a 5′-OH group of a support bound ribonucleoside or oligoribonucleotide with a monomeric ribonucleoside phosphoramidite having a Silyl-ELgp-protected hydroxyl group, to provide an intermediate in which the support-bound ribonucleoside or oligoribonucleotide is bound to the monomeric ribonucleoside through a phosphate triester linkage; (b) treating the intermediate provided in step (a) with a deprotecting reagent effective to convert the Silyl-ELgp-protected hydroxyl group to a free hydroxyl moiety; and, c) repeating steps (a)-(b) until the desired sequence of RNA is obtained. Any suitable condition can be used to remove the Silyl-ELgp protecting groups. In some embodiments, solutions containing TBAF (TetraButylAmonium Fluoride) can be used. In some embodiments, HF/TEMED or HF/TEA (TriethylAmine) can be used. In some embodiments, there are provided herein methods of synthesizing a sequence of RNA, the method comprising the steps of: (a) condensing a 3′-OH or a 5′-OH group of a support bound ribonucleoside or oligoribonucleotide with a monomeric ribonucleoside phosphoramidite having a Silyl-ELgp-protected hydroxyl group, to provide an intermediate in which the support-bound ribonucleoside or olioribonucleotide is bound to the monomeric ribonucleoside through a phosphate triester linkage, and (b) treating the intermediate provided in step (a) with a deprotecting reagent effective to convert the Silyl-ELgp-protected hydroxyl group to a free hydroxyl moiety and simultaneously oxidize the phosphate triester linkage to give a phosphotriester linkage. Non-limiting examples of such deprotecting reagent comprise: HF/TEA/ROOH; HF/TEMED/ROOH; and TBAF/ROOH.

Silyl-ELgp protection on the 5′hydroxyl (or 3′ hydroxyl) as described herein allows synthesis of long sequences of RNA which were not possible to chemically synthesize before. In some embodiments, an oligoribonucleotide synthesized by methods disclosed herein is at least 20, 25, 30, 35, 40, 45, 50, 55, 60, 65, 70, 75, 80, 85, 90, 95, 100, 105, 110, 115, 120, 125, 130, 135, 140, 145, 150, 300, 500, 1000 or more nucleotides in length. Furthermore, an oligoribonucleotide synthesized as described herein can be combined with another oligoribonucleotide to form a longer oligoribonucleotide. For example, an oligoribonucleotide of 70 bases can be coupled with another oligoribonucleotide of 70 bases by chemical ligation. As another example, two oligoribonucleotides can be ligated with an RNA ligase. In this case, the 5′-protecting groups can be removed before ligation.

The synthetic methods as described herein can be conducted on a solid support having a surface to which chemical entities may bind. In some embodiments, multiple oligoribonucleotides being synthesized are attached, directly or indirectly, to the same solid support and can form part of an array. In some embodiments, oligoribonucleotides being synthesized are attached to a bead directly or indirectly. Suitable solid supports may have a variety of forms and compositions and derive from naturally occurring materials, naturally occurring materials that have been synthetically modified, or synthetic materials. Examples of suitable support materials include, but are not limited to, silicas, teflons, glasses, polysaccharides such as agarose (e.g., Sepharose® from Pharmacia) and dextran (e.g., Sephadex® and Sephacyl®, also from Pharmacia), polyacrylamides, polystyrenes, polyvinyl alcohols, copolymers of hydroxyethyl methacrylate and methyl methacrylate, and the like. The initial monomer of the oligoribonucleotide to be synthesized on the substrate surface is typically bound to a linking moiety which is in turn bound to a surface hydrophilic group, e.g., a surface hydroxyl moiety present on a silica substrate. In some embodiments, a universal linker is used. In some other embodiments, the initial monomer is reacted directly with, e.g., a surface hydroxyl moiety. Alternatively, oligoribonucleotides can be synthesized first according to the present disclosure, and attached to a solid substrate post-synthesis by any method known in the art. Thus, the present methods can be used to prepare arrays of oligoribonucleotides wherein the oligoribonucleotides are either synthesized on the array, or attached to the array substrate post-synthesis.

With the efficiency and ease of the present methods, oligoribonucleotide synthesis can be performed in small or large scales. The quantity of oligoribonucleotide made in one complete run of the present methods (in one container) can thus be less than a microgram, or in micrograms, tens of micrograms, hundreds of micrograms, grams, tens of grams, hundreds of grams, or even kilograms.

### Compositions of Matter

The ability to introduce a Silyl-ELgp protecting group using an activated carbonate or activated S-thiocarbonate such as chloroformate or thiochloroformate, in general leads to high yield synthesis of the 5′-hydroxyl (or 3′-hydroxyl) protected monomers. Other activating groups include, for example, p-nitrophenoxy, O-succinimidyl, trichloromethyl, bromo and iodo. Active carbonates and thiocarbonates such as thiochloroformates and chloroformates are also reactive with, and provide stable protection of, the exocyclic amine groups of the heterobases. Synthesis of the RNA phosphoramidites therefore becomes very straightforward and easy, as well as very cost efficient. The high reaction yield of chloroformates or thiochloroformates can reduce the cost of RNA precursor monomers.

In some embodiments, protected nucleoside monomers are provide herein comprising compositions of matter useful, inter alia, in the synthesis of oligoribonucleotides as described herein. In some embodiments, there are provided nucleoside monomers having the following structure:

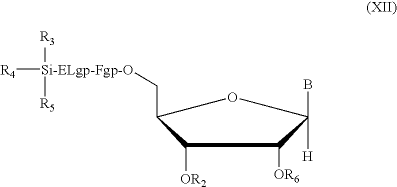

wherein B is a protected or non-protected heterocycle;
R₂ is selected from H, a protecting group, and a phosphoramidite group;
each of R₃, R₄, R₅ is independently selected from hydrocarbyls, substituted hydrocarbyls, aryls, and substituted hydrocarbyls; and wherein ELgp is an eliminating group as described herein.

In some embodiments, the monomers have the following formula:

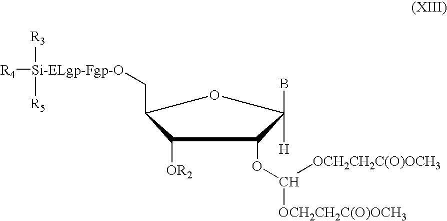

In some embodiments, the monomers have the following formula:

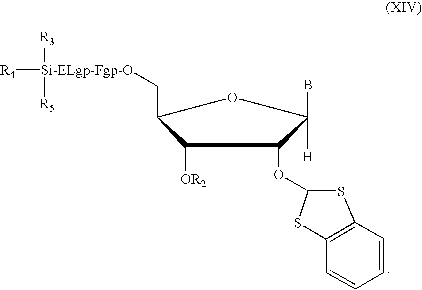

In some embodiments, the monomers have the structural formulae (II) and (X) described above.

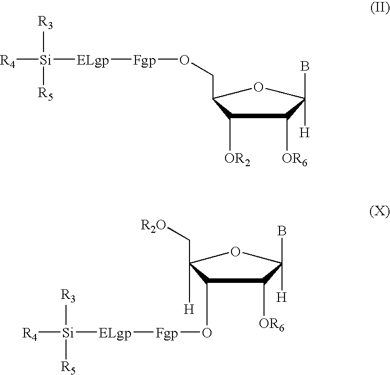

R₂ represents a phosphorus derivative that enables coupling to a free hydroxyl group, and comprises a phosphoramidite having the structure (III)

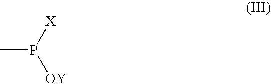

wherein X and Y are as defined earlier herein.

In some embodiments, a reagent of formula (II), used for 3′-to-5′ synthesis, can be prepared by reaction of the unprotected ribonucleoside with the haloformate

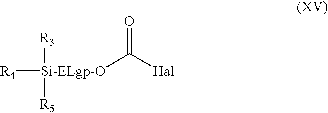

wherein Hal represents halo, typically chloro, in the presence of a base effective to catalyze the nucleophilic reaction, e.g., pyridine. This step results in a protected ribonucleotide derivative, as follows:

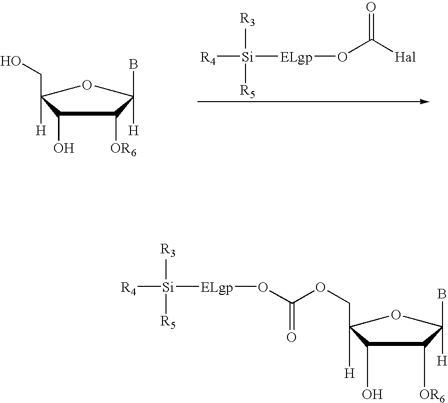

The intermediate so prepared can then be phosphitylated with the phosphoramidite PX₂(OY) wherein X and Y are as defined earlier, resulting in conversion of the 3′-hydroxyl moiety to the desired substituent —O—PX(PY):

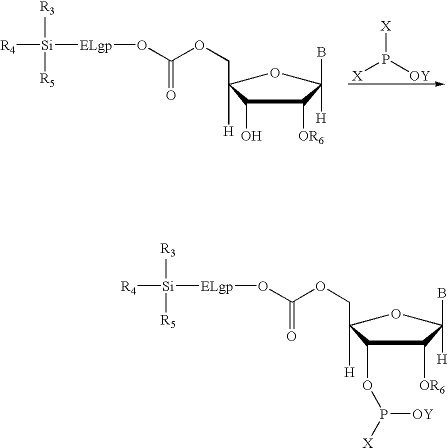

In some embodiments, reagent (X), used for 5′-to-3′ synthesis, may be prepared by first synthesizing a 5′-protected nucleoside using a conventional 5′-OH protecting group such as DMT. This 5′-protected nucleoside is then reacted with the haloformate

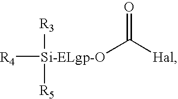

which, as above, is done in the presence of a base effective to catalyze the nucleophilic reaction, e.g., pyridine. The DMT group is then removed with acid, resulting in the 3′-Silyl-ELgp intermediate:

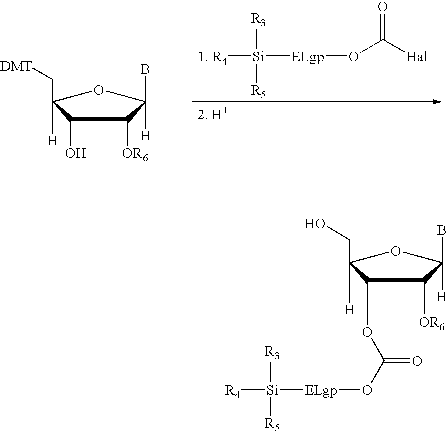

Subsequent reaction with the phosphoramidite results in conversion of the 5′-hydroxyl moiety to the desired substituent —O—PX(YO), i.e., —OR₂:

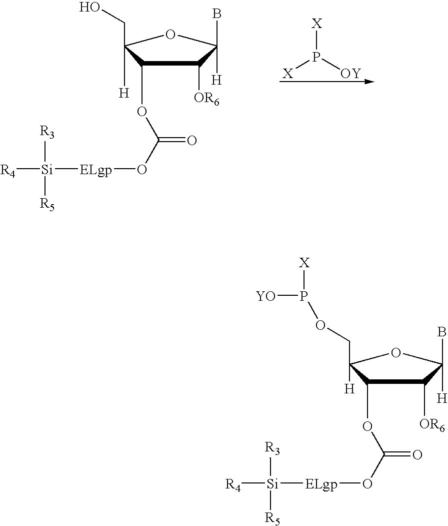

Analogous Silyl-ELgp-S—C(O)-5′OH (or 3′OH)-protected monomers can be prepared in similar reactions carried out using the following haloformate:

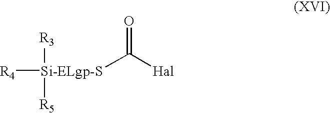

### Kits

In some embodiments there are provided various kits for RNA synthesis. In some embodiments, the kits comprise at least one ribonucleoside monomer having the structure of Formula I. In some embodiments, kits can comprise at least one monomer selected from Formula I, Formula II, Formula III, Formula IX, Formula X, Formulat XII, Formulat XIII and Formulat XIV. Some embodiments comprise at least one of four such ribonucleoside monomers, comprising adenine, uracil, guanine, and cytosine, respectively. Each of the adenine, guanine, and cytosine is optionally protected, preferably by the same Silyl-ELgp protecting group protecting the 5′-OH (or 3′-OH) of the ribonucleoside. The ribonucleoside monomers optionally comprise other-protecting groups, and/or a phosphoramidite group. The kits may comprise reagents for post-synthesis RNA deprotection, as described herein, such as, for example, TBAF, tBuOOH, H₂O₂, HF, HF-pyridine, HF-TEMED HF-TEA, pyridine, TEMED, and/or TEA.

In some embodiments, kits comprise components useful for the preparation of ribonucleoside monomer precursors. Kits may comprise TIPSCl₂ and thiochloroformates comprising the structure such as, e.g., Cl—CO—S-ELgp-Si—R³R⁴R⁵ or oxychloroformates comprising Cl—CO—O-ELgp-Si—R³R⁴R⁵. Kits may further comprise reagents such as HF, pyridine, CH₃CN, DMT-containing blocking agents (such as DMT chloride), and/or CH₃OP(NiPr₂)₂. Kits may also comprise unprotected nucleoside monomers, such as adenosine, guanosine, uridine and/or cytidine.

### Synthesis of Oligonucleotide Arrays

In some embodiments, there are provided herein methods for making an oligoribonucleotide array made up of array features each presenting a specified oligoribonucleotide sequence at an address on an array substrate. First, the array substrate is treated to protect the hydroxyl moieties on the derivatized surface from reaction with phosphoramidites or analogous phosphorus groups used in oligoribonucleotide synthesis. Protection can involve conversion of free hydroxyl groups to Silyl-ELgp-Fgp-protected groups. The methods then involve (a) applying droplets of an alpha effect nucleophile to effect deprotection of hydroxyl moieties at selected addresses and oxidation of the newly formed internucleotide phosphite triester linkages, followed by (b) flooding the array substrate with a medium containing a selected ribonucleoside monomer having the structure of either Formula (II) (for 3′-to-5′ synthesis) or Formula (X) (for 5′-to-3′ synthesis). Step (a), deprotection/oxidation, and step (b), monomer addition, are repeated to sequentially build oligonucleotides having the desired sequences at selected addresses to complete the array features. In a variation on the aforementioned method, the applied droplets may comprise the selected ribonucleoside monomer, while the alpha effect nucleophile is used to flood the array substrate; that is, steps (a) and (b) are essentially reversed.

In some embodiments of the array construction methods, the deprotection reagents are aqueous, allowing for good droplet formation on a wide variety of array substrate surfaces. Moreover, because the selection of features employs aqueous media, small-scale discrete droplet application onto specified array addresses can be carried out by adaptation of techniques for reproducible fine droplet deposition from printing technologies.

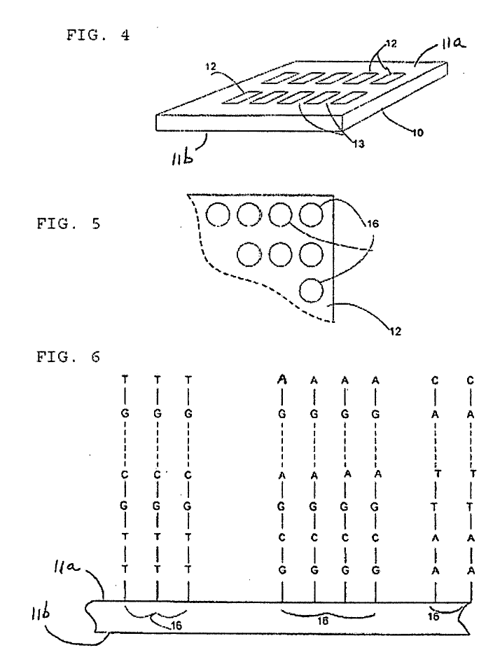

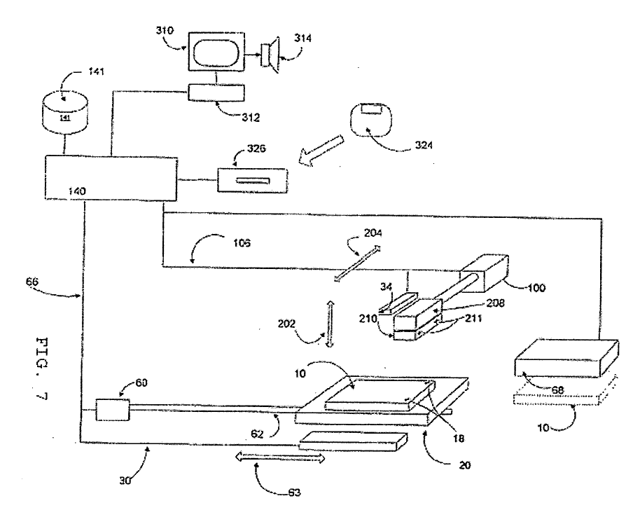

Referring now to FIGS. 4 through 6, some embodiments of the present methods can be used to produce multiple identical arrays 12 (only some of which are shown in FIG. 4) across a complete front surface 11a of a single substrate 10 (which also has a back surface 11b). However, the arrays 12 produced on a given substrate need not be identical and some or all could be different. Each array 12 will contain multiple spots or features 16. The arrays 12 are shown as being separated by spaces 13. A typical array 12 may contain from 100 to 100,000 features. All of the features 16 can be different, or some or all could be the same. Each feature carries a predetermined polynucleotide having a particular sequence, or a predetermined mixture of polynucleotides. This is illustrated schematically in FIG. 6 where different regions 16 are shown as carrying different polynucleotide sequences. While arrays 12 are shown separated from one another by spaces 13, and the features 16 are separated from one another by spaces, such spaces in either instance are not essential.

In a typical execution of the present methods, a polynucleotide is synthesized using one or more nucleoside phosphoramidites in one or more synthesis cycles having a) a coupling step, and b) a concurrent oxidation/deprotection step using the combined oxidation/deprotection reagent, as described above (with optional capping). In particular, the fabrication of each array 12 will be described. It will first be assumed that a substrate bound moiety is present at least at the location of each feature or region to be formed (that is, at each address). Such substrate bound moiety may, for example, be a nucleoside monomer which was deposited and deprotected at the location of each feature in a previous cycle, such that the deprotected reactive site hydroxyl is available for linking to another activated nucleoside monomer. Alternatively, the substrate bound moiety may be a suitable linking group previously attached to substrate 10. Both of these steps are known in in situ fabrication techniques. A droplet of a nucleoside phosphoramidite monomer solution is deposited onto the address and activated with a suitable activator (for example, a tetrazole, an imidazole, nitroimidazole, benzimidazole and similar nitrogen heterocyclic proton donors). In the case of phosphoramidites a non-protic low boiling point solvent could be used, for example, acetonitrile, dioxane, toluene, ethylacetate, acetone, tetrahydrofuran, and the like. Suitable activators for phosphoramidites are known and include tetrazole, S-ethyl tetrazole, dicyanoimidazole (“DCI”), or benzimidazolium triflate.

Any suitable droplet deposition technique, such as a pulse jet (for example, an inkjet head) may be used. The nucleoside phosphoramidite may particularly be of formula (II) with R₂ being of formula (III) where Y is cyanoethyl, X is N(isopropyl)₂. Alternatively, a Silyl-ELgp-Fgp protective group could be on the 3′ carbon and the phosphoramidyl group on the 5′ carbon, if it was desired to have the polynucleotide grow in the 5′ to 3′ direction. The activated phosphoramidyl group will then couple the nucleoside monomer through a corresponding phosphite linkage with the substrate bound moiety (again, a linking group previously attached to substrate 10 or a deprotected nucleoside monomer deposited in a previous cycle). Note that the phosphite linkage corresponding to the foregoing particular phosphoramidite will be as in formula (IV) above. Particularly in the case of phosphoramidites, the reaction is complete very rapidly at room temperature of about 20° C. (for example, in one or two seconds).

At this point, a capping of substrate bound reactive site hydroxyls which failed to couple with a nucleoside compound may optionally be performed using known procedures.

The resulting compound can then be reacted with the combined oxidation/ deprotection reagent composition. In some embodiments, such a composition can oxidize the phosphite linkage at a rate which is greater than the deprotection rate. In manufacture of a typical array, suitable times for exposure of the substrate to such solutions may range from about 10 to 60 seconds followed by washing with a non-aqueous solvent for about 10 to 60 seconds: Suitable solvents include aromatic solvents (such as benzene, xylene and particularly toluene) as well as chlorinated hydrocarbons (particularly chlorinated lower alkyl hydrocarbons such as dichloromethane).

The above steps can be repeated at each of many addresses on substrate 10 until the desired polynucleotide at each address has been synthesized. It will be understood however, that intermediate, washing and other steps may be required between cycles, as is well known in the art of synthesizing polynucleotides. Note though that since oxidation and deprotection are accomplished with a single composition, no washes are required between such steps. Furthermore, as water may optionally be substantially eliminated, the thorough washing to remove water prior to the coupling step in the next cycle is not required or may be reduced. The cycles may be repeated using different or the same biomonomers, at multiple regions over multiple cycles, as required to fabricate the desired array or arrays 12 on substrate 10. Note that oxidation and deprotection is preferably performed by exposing substrate 10 (in particular, the entire first surface 11a) to the single combined oxidation/deprotection reagent composition, for example, by flowing such a solution across first surface 11a. When all cycles to form the desired polynucleotide sequences at all addresses on the array have been completed, the substrate is dipped into a 1:1 solution of a 40% methylamine in water and 28% ammonia in water. This solution removes the protecting groups on the phosphate linkages and on the purine or pyrimidine base exocyclic amine functional groups. The arrays may then be removed from the solution and washed with water and are ready for use.

Referring now to FIG. 7, some embodiments, of suitable apparatuses for fabricating polynucleotide arrays in accordance with the present disclosure is shown. The apparatuses shown include a substrate station 20 on which can be mounted a substrate 10. Pins or similar means (not shown) can be provided on substrate station 20 by which to approximately align substrate 10 to a nominal position thereon. Substrate station 20 can include a vacuum chuck connected to a suitable vacuum source (not shown) to retain a substrate 10 without exerting too much pressure thereon, since substrate 10 is often made of glass. A flood station 68 is provided which can expose the entire surface of substrate 10, when positioned beneath station 68 as illustrated in broken lines in FIG. 7, to a fluid typically used in the in situ process, and to which all features can be exposed during each cycle.

A dispensing head 210 is retained by a head retainer 208. The positioning system includes a carriage 62 connected to a first transporter 60 controlled by processor 140 through line 66, and a second transporter 100 controlled by processor 140 through line 106. Transporter 60 and carriage 62 are used execute one axis positioning of station 20 (and hence mounted substrate 10) facing the dispensing head 210, by moving it in the direction of arrow 63, while transporter 100 is used to provide adjustment of the position of head retainer 208 (and hence head 210) in a direction of axis 204. In this manner, head 210 can be scanned line by line, by scanning along a line over substrate 10 in the direction of axis 204 using transporter 100, while line by line movement of substrate 10 in a direction of axis 63 is provided by transporter 60. Transporter 60 can also move substrate holder 20 to position substrate 10 beneath flood station 68 (as illustrated in broken lines in FIG. 7). Head 210 may also optionally be moved in a vertical direction 202, by another suitable transporter (not shown). It will be appreciated that other scanning configurations could be used. It will also be appreciated that both transporters 60 and 100, or either one of them, with suitable construction, could be used to perform the foregoing scanning of head 210 with respect to substrate 10. Thus, when the present application recites “positioning” one element (such as head 210) in relation to another element (such as one of the stations 20 or substrate 10) it will be understood that any required moving can be accomplished by moving either element or a combination of both of them. The head 210, the positioning system, and processor 140 together act as the deposition system of the apparatus. An encoder 30 communicates with processor 140 to provide data on the exact location of substrate station 20 (and hence substrate 10 if positioned correctly on substrate station 20), while encoder 34 provides data on the exact location of holder 208 (and hence head 210 if positioned correctly on holder 208). Any suitable encoder, such as an optical encoder, may be used which provides data on linear position.

Head 210 may be of a type commonly used in an ink jet type of printer and may, for example, include five or more chambers (at least one for each of four nucleoside phosphoramidite monomers plus at least one for a solution of solid activator) each communicating with a corresponding set of multiple drop dispensing orifices and multiple ejectors which are positioned in the chambers opposite respective orifices. Each ejector is in the form of an electrical resistor operating as a heating element under control of processor 140 (although piezoelectric elements could be used instead). Each orifice with its associated ejector and portion of the chamber, defines a corresponding pulse jet. It will be appreciated that head 210 could, for example, have more or less pulse jets as desired (for example, at least ten or at least one hundred pulse jets). Application of a single electric pulse to an ejector will cause a droplet to be dispensed from a corresponding orifice. Certain elements of the head 210 can be adapted from parts of a commercially available thermal inkjet print head device available from Hewlett-Packard Co. as part no. HP51645A. Alternatively, multiple heads could be used instead of a single head 210, each being similar in construction to head 210 and being provided with respective transporters under control of processor 140 for independent movement. In this alternate configuration, each head may dispense a corresponding biomonomer (for example, one of four nucleoside phosphoramidites) or a solution of a solid activator.

As is well known in the ink jet print art, the amount of fluid that is expelled in a single activation event of a pulse jet, can be controlled by changing one or more of a number of parameters, including the orifice diameter, the orifice length (thickness of the orifice member at the orifice), the size of the deposition chamber, and the size of the heating element, among others. The amount of fluid that is expelled during a single activation event is generally in the range about 0.1 to 1000 pL, usually about 0.5 to 500 pL and more usually about 1.0 to 250 pL. A typical velocity at which the fluid is expelled from the chamber is more than about 1 m/s, usually more than about 10 m/s, and may be as great as about 20 m/s or greater. As will be appreciated, if the orifice is in motion with respect to the receiving surface at the time an ejector is activated, the actual site of deposition of the material will not be the location that is at the moment of activation in a line-of-sight relation to the orifice, but will be a location that is predictable for the given distances and velocities.

The apparatuses can deposit droplets to provide features which may have widths (that is, diameter, for a round spot) in the range from a minimum of about 10 mu.m to a maximum of about 1.0 cm. In embodiments where very small spot sizes or feature sizes are desired, material can be deposited according to the apparatuese in small spots whose width is in the range about 1.0 μm to 1.0 mm, usually about 5.0 μm to 500 μm, and more usually about 10 μm to 200 μm.

The apparatuses can include a display 310, speaker 314, and operator input device 312. Operator input device 312 may, for example, be a keyboard, mouse, or the like. Processor 140 has access to a memory 141, and controls print head 210 (specifically, the activation of the ejectors therein), operation of the positioning system, operation of each jet in print head 210, and operation display 310 and speaker 314. Memory 141 may be any suitable device in which processor 140 can store and retrieve data, such as magnetic, optical, or solid state storage devices (including magnetic or optical disks or tape or RAM, or any other suitable device, either fixed or portable). Processor 140 may include a general purpose digital microprocessor suitably programmed from a computer readable medium carrying necessary program code, to execute all of the steps required by the present methods, or any hardware or software combination which will perform those or equivalent steps. The programming can be provided remotely to processor 141, or previously saved in a computer program product such as memory 141 or some other portable or fixed computer readable storage medium using any of those devices mentioned below in connection with memory 141. For example, a magnetic or optical disk 324 may carry the programming, and can be read by disk reader 326.

Operation of the apparatus of FIG. 7 in accordance with some embodiments of the present methods will now be described. First, it will be assumed that memory 141 holds a target drive pattern. This target drive pattern is the instructions for driving the apparatus components as required to form the target array (which includes target locations and dimension for each spot) on substrate 10 and includes, for example, movement commands to transporters 60 and 100 as well as firing commands for each of the pulse jets in head 210 coordinated with the movement of head 210 and substrate 10. This target drive pattern is based upon the target array pattern and can have either been input from an appropriate source (such as input device 312, a portable magnetic or optical medium, or from a remote server, any of which communicate with processor 140), or may have been determined by processor 140 based upon an input target array pattern (using any of the appropriate sources previously mentioned) and the previously known nominal operating parameters of the apparatus. The target drive pattern further includes instructions to head 210 and the positioning system of the apparatus to deposit the solution of solid activator at each region at which a biomonomer is to be deposited, separate from and preceding deposition of the biomonomer. Further, it will be assumed that each of four chambers of head 210 has been loaded with four different nucleoside phosphoramidite monomers, while a fifth chamber has been loaded with activating agent. It will also be assumed that flood station 68 has been loaded with all necessary solutions. Operation of the following sequences are controlled by processor 140, following initial operator activation, unless a contrary indication appears.

For any given substrate 10, the operation is basically as follows, assuming in situ preparation of a typical oligonucleotide using standard nucleoside phosphoramidite monomers as the biomonomers. A substrate 10 is loaded onto substrate station 20 either manually by an operator, or optionally by a suitable automated driver (not shown) controlled, for example, by processor 140. A target drive pattern necessary to obtain a target array pattern, is determined by processor 140 (if not already provided), based on nominal operating parameters of the apparatus. The apparatus is then operated as follows: (a) dispense appropriate next nucleoside phosphoramidite onto each region such that the first linking group is activated by solid activator and links to previously deposited deprotected nucleoside monomer; (b) move substrate 10 to flood station 68 for exposure to single combined oxidation/deprotection reagent composition as described herein, and washing solution, as well as optional capping solution, all over entire substrate as required; and (e) repeat foregoing cycle for all the regions of all desired arrays 12 until the desired arrays are completed (note that the biomonomer deposited and linked to the substrate bound moiety in one cycle becomes the substrate bound moiety for the next cycle). The phosphoramidite solution may include an activator, or alternatively a separate solid activator may be formed in the manner described in U.S. patent application Ser. No. 09/356,249, filed Jul. 16, 1999 and entitled “Biopolymer Arrays and Their Fabrication”, incorporated herein by reference.

Note that during the above operation, pressure within head 210 can be controlled as described in patent applications “FABRICATING BIOPOLYMER ARRAYS”, by Caren et al., Ser. No. 09/302,922, and “PREPARATION OF BIOPOLYMER ARRAYS” by A. Schleifer et al., Ser. No. 09/302,899, now U.S. Pat. No. 6,242,266, both filed Apr. 30, 1999 and both assigned to the same assignee as the present application, and the references cited therein. Processor 140 can execute the control of pressure within head 210.

With regard to the actual deposition sequence of biomonomer or activator solution droplets, as already mentioned, in this sequence processor 140 will operate the apparatus according to the target drive pattern, by causing the positioning system to position head 210 facing substrate station 20, and particularly the mounted substrate 10, and with head 210 at an appropriate distance from substrate 10. Processor 140 then causes the positioning system to scan head 210 across substrate 14 line by line (or in some other desired pattern), while co-ordinating activation of the ejectors in head 210 so as to dispense droplets in accordance with the target pattern. This can be continued until all arrays 12 to be formed on substrate 10 have been completed. The number of spots in any one array 12 can, for example, be at least ten, at least one hundred, at least one thousand, or even at least one hundred thousand.

At this point the droplet dispensing sequence is complete.

Arrays fabricated by methods and apparatus described herein, can be used to evaluate for the presence of one or more target polynucleotides in a known manner. Basically, this involves exposing the sample, normally as a fluid composition, to the array, such that target polynucleotide which may be present will bind to one or more predetermined regions of the array. The binding pattern on the array may then be observed by any method (such as by observing a fluorescence pattern), and the presence of the target evaluated based, in whole or in part, on the observed binding pattern.

Modifications in the particular embodiments described above are, of course, possible. For example, where a pattern of arrays is desired, any of a variety of geometries may be constructed other than the organized rows and columns of arrays 12 of FIG. 4. For example, arrays 12 can be arranged in a series of curvilinear rows across the substrate surface (for example, a series of concentric circles or semi-circles of spots), and the like. Similarly, the pattern of regions 16 may be varied from the organized rows and columns of spots in FIG. 4 to include, for example, a series of curvilinear rows across the substrate surface(for example, a series of concentric circles or semi-circles of spots), and the like. Even irregular arrangements of the arrays or the regions within them can be used, at least when some means is provided such that during their use the locations of regions of particular characteristics can be determined (for example, a map of the regions is provided to the end user with the array).

The present methods and apparatus may be used to form arrays of polyribonucleotides or other polymers made of monomers having a hydroxy protecting group and which are initially linked through a phosphite group (which is then oxidized) on surfaces of any of a variety of different substrates, including both flexible and rigid substrates. Preferred materials provide physical support for the deposited material and endure the conditions of the deposition process and of any subsequent treatment or handling or processing that may be encountered in the use of the particular array. The array substrate may take any of a variety of configurations ranging from simple to complex. Thus, the substrate could have generally planar form, as for example a slide or plate configuration, such as a rectangular or square or disc. In many embodiments, the substrate will be shaped generally as a rectangular solid, having any desired dimensions, such as a length in the range about 4 mm to 500 mm; a width in the range about 4 mm to 500 mm. However, larger substrates can be used, particularly when such are cut after fabrication into smaller size substrates carrying a smaller total number of arrays 12. Substrates of other configurations and equivalent areas can be chosen. The configuration of the array may be selected according to manufacturing, handling, and use considerations.

The substrates may be fabricated from any of a variety of materials. In certain embodiments, such as for example where production of binding pair arrays for use in research and related applications is desired, the materials from which the substrate may be fabricated should ideally exhibit a low level of non-specific binding during hybridization events. In many situations, it will also be preferable to employ a material that is transparent to visible and/or UV light. For flexible substrates, materials of interest include: nylon, both modified and unmodified, nitrocellulose, polypropylene, and the like, where a nylon membrane, as well as derivatives thereof, may be particularly useful in this embodiment. For rigid substrates, specific materials of interest include: glass; fused silica, silicon, plastics (for example, polytetrafluoroethylene, polypropylene, polystyrene, polycarbonate, and blends thereof, and the like); metals (for example, gold, platinum, and the like).

The substrate surface onto which the polynucleotide compositions or other moieties are deposited may be smooth or substantially planar, or have irregularities, such as depressions or elevations. The surface may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero- or homopolymeric, and may or may not have separate functional moieties attached thereto (for example, conjugated).

It is to be understood that while the invention has been described in conjunction with some embodiments thereof, that the description above as well as the example which follows are intended to illustrate and not limit the scope of the invention. Other aspects, advantages and modifications within the scope of the invention will be apparent to those skilled in the art to which the invention pertains.

The practice of the present invention will employ, unless otherwise indicated, conventional techniques of synthetic organic chemistry, biochemistry, molecular biology, and the like, which are within the skill of the art. Such techniques are explained fully in the literature.

All patents, patent applications, journal articles and other references mentioned herein are incorporated by reference in their entireties.

What is claimed is:

1. A ribnucleoside monomer having the structure of Formula (I): 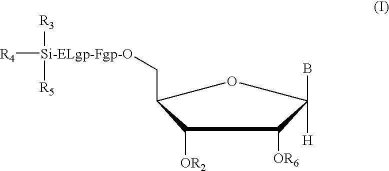
wherein B is a protected or non-protected heterocycle;
R₂ is selected from H, a protecting group, and a phosphoramidite group;
each of R₃, R₄, R₅ is independently selected from hydrocarbyls, substituted hydrocarbyls, aryls, and substituted hydrocarbyls; wherein ELgp is an eliminating group, wherein ELgp is not oxygen-linked or sulfur-linked to the Si atom; wherein R₆ is a protecting group;
wherein Fgp is an optional linking group selected from oxycarbonyl (O—C(O)), and thiocarbonyl (S—C(O)), and
wherein ELgp is not oxygen-linked or sulfur-linked to the Si atom.

2. The ribonucleoside monomer of claim 1, wherein ELgp is selected from the group consisting of ethylene, substituted ethylene, —(CH₂CH₂O)ₙ—, substituted —(CH₂CH₂O)ₙ—, —(CH₂CH₂O)ₙ—CH₂CH₂—, substituted —(CH₂CH₂O)ₙ—CH₂CH₂— (wherein n is an integer from 1 to 8), and the following functional groups (in the direction from Fgp to Si), and any repeats and combinations of said functional groups: 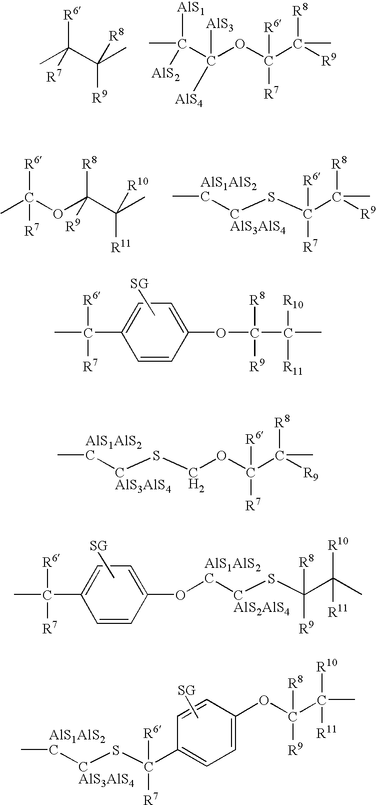
wherein each of R^{6′}, R⁷, R⁸, R⁹, R¹⁰, and R¹¹ is independently selected from the group consisting of H, hydrocarbyls, substituted hydrocarbyls, aryls, and substituted aryls; each AIS is a substituent allowable for episulfide formation; SG is one or multiple substituents on the phenyl ring independently selected from the group consisting of H, hydrocarbyls, substituted hydrocarbyls, aryls, and substituted aryls; and wherein R₆ is one of the following structures: 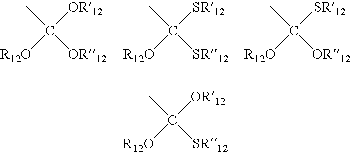
wherein each of R₁₂, R′₁₂, and R″₁₂ are independently selected from lower hydrocarbyls, substituted-lower hydrocarbyls, aryls and substituted hydrocarbyls, wherein R₁₂ is optionally H, and wherein R′₁₂ and R″₁₂ are optionally cyclically connected.

3. The ribonucleoside monomer of claim 1, wherein at least one of R₃, R₄, and R₅ is a lower alkyl.

4. The ribonucleoside monomer of claim 1, wherein at least one of R₃, R₄, and R₅ is an aryl.

5. The ribonucleoside monomer of claim 1, wherein each of R₃, R₄, and R₅ comprises a phenyl group or substituted phenyl group.

6. The ribonucleoside monomer of claim 1 having a formula comprising:

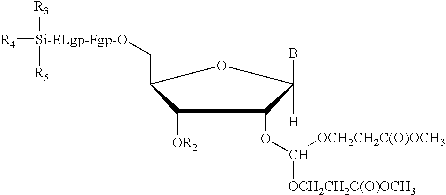

7. The nucleoside monomer of claim 1 having a formula comprising:

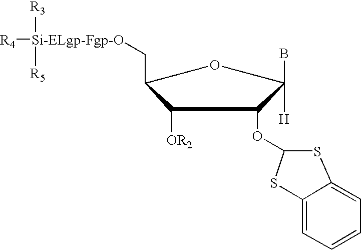

8. A method for synthesizing a ribonucleoside monomer, comprising synthesizing the ribonucleoside monomer according to Scheme I.

9. A method of synthesizing a sequence of RNA, the method comprising the steps of:
(a) condensing a 3′-OH or a 5′-OH group of a support bound ribonucleoside or oligoribonucleotide with a monomeric ribonucleoside phosphoramidite having a Silyl-ELgp-protected hydroxyl group, to provide an intermediate in which the support-bound ribonucleoside or oligoribonucleotide is bound to the monomeric ribonucleoside through a phosphate triester linkage;
(b) treating the intermediate provided in step (a) with a deprotecting reagent effective to convert the Silyl-ELgp-protected hydroxyl group to a free hydroxyl moiety; and,
c) repeating steps (a)-(b) until the desired sequence of RNA is obtained.

10. The method of claim 9 wherein the deprotecting reagent comprises at least one of HF/pyridine, HF/TEA, HF/TEMED, and TBAF.

11. A method of synthesizing a sequence of RNA, the method comprising the steps of:
(a) condensing a 3′-OH or a 5′-OH group of a support bound ribonucleoside or oligoribonucleotide with a monomeric ribonucleoside phosphoramidite having a Silyl-ELgp-protected hydroxyl group, to provide an intermediate in which the support-bound ribonucleoside or oligoribonucleotide is bound to the monomeric ribonucleoside through a phosphate triester linkage;
(b) treating the intermediate provided in step (a) with a deprotecting reagent effective to convert the Silyl-ELgp-protected hydroxyl group to a free hydroxyl moiety and simultaneously oxidize the phosphate triester linkage to give a phosphotriester linkage; and,
c) repeating steps (a)-(b) until the desired sequence of RNA is obtained.

12. The method of claim 11 wherein the deprotecting reagent comprises at least one of HF/TEA/ROOH, HF/TEMED/ROOH, and TBAF/ROOH.

13. A method for synthesizing a riboligonucleotide, the method comprising the steps of:
(a) providing a ribonucleoside having the following structure: 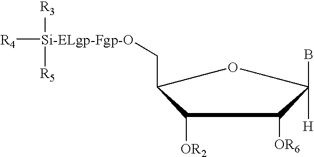
wherein B is a protected or non-protected heterocycle;
R₂ is a phosphoramidite group;
each of R₃, R₄, R₅ is independently selected from hydrocarbyls, substituted hydrocarbyls, aryls, and substituted hydrocarbyls; wherein ELgp is an eliminating group, wherein ELgp is not oxygen-linked or sulfur-linked to the Si atom; and
wherein Fgp is an optional linking group selected from oxycarbonyl (O—C(O)), and thiocarbonyl (S—C(O));
(b) coupling the ribonucleoside with a second ribonucleoside or an oligoribonucleotide, wherein the 3′-end of the second ribonucleoside or oligoribonucleotide is bound directly or indirectly to a solid support, and said second ribonucleoside or oligoribonucleotide has a free 5′-OH group; and
(c) deprotecting with fluoride ion.

14. A method for synthesizing an oligoribonucleotide, comprising:
(a) providing a ribonucleoside having the following structure: 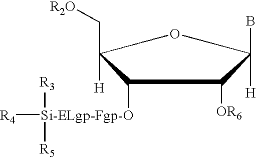
wherein B is a protected or non-protected heterocycle;
R₂ is a phosphoramidite group;
each of R₃, R₄, R₅ is independently selected from hydrocarbyls, substituted hydrocarbyls, aryls, and substituted hydrocarbyls; wherein ELgp is an eliminating group, wherein ELgp is not oxygen-linked or sulfur-linked to the Si atom; and
wherein Fgp is an optional linking group selected from oxycarbonyl (O—C(O)), and thiocarbonyl (S—C(O));
(b) coupling the ribonucleoside with a second ribonucleoside or an oligoribonucleotide, wherein the 5′-end of the second ribonucleoside or oligoribonucleotide is bound directly or indirectly to a solid support, and said second ribonucleoside or oligoribonucleotide has a free 3′-OH group; and
(c) deprotecting with fluoride ion.

15. The method of any one of claims 13-14, wherein at least one of R₃, R₄, and R₅ is an aryl.

16. The method of any one of claims 13-14, wherein the oligoribonucleotide being synthesized and the solid support are part of an array.

17. The method of any one of claims 13-14, wherein the oligoribonucleotide is synthesized in a quantity of grams.

18. The method of any one of claims 13-14, wherein the oligoribonucleotide is synthesized in a quantity of kilograms.

19. The method of any one of claims 13-14, wherein the oligoribonucleotide is at least about 100 nucleotides in length.

20. A kit for RNA synthesis, comprising four ribonucleoside monomers according to claim 1, wherein the B moieties in the four ribonucleoside monomers are adenine, guanine, uracil and cytosine, respectively, or protected counterparts thereof.

21. A kit for synthesizing a nucleoside monomer precursor comprising an alpha-effect nucleophile and a haloformate of the following structure: 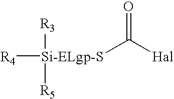
wherein:
each of R₃, R₄, R₅ is independently selected from hydrocarbyls, substituted hydrocarbyls, aryls and substituted hydrocarbyls; and wherein ELgp is an eliminating group.

22. A method for making an oligoribonucleotide array made up of array features each presenting a specified oligoribonucleotide sequence at an address on an array substrate, the method comprising steps of: providing a hydroxyl-derivatized array substrate and treating the array substrate to protect hydroxyl moieties on the derivatized surface from reaction with phosphoramidites, then iteratively carrying out the steps of (i) applying droplets of an alpha effect nucleophile to effect deprotection of hydroxyl moieties at selected addresses, and (ii) flooding the array substrate with a medium containing a selected monomeric ribonucleoside of claim 1, to permit covalent attachment of the selected ribonucleoside to the deprotected hydroxyl moieties at the selected addresses.
